(19) United States
(12) Patent Application Publication
(10) Pub. No.: US 2024/0282416 A1
(43) Pub. Date: Aug. 22, 2024

(54) SYSTEMS AND METHODS FOR LEARNING PATIENT EMBEDDINGS WITH AUTOENCODERS

(71) Applicant: CADENCE SOLUTIONS, INC., New York, NY (US)

(72) Inventor: Ashwyn SHARMA, Seattle, WA (US)

(73) Assignee: CADENCE SOLUTIONS, INC., New York, NY (US)

(21) Appl. No.: 18/171,044

(22) Filed: Feb. 17, 2023

(51) Int. Cl. G16H 50/20; G06N 3/0455

(52) U.S. Cl. CPC G16H 10/60; G06N 3/0455; G16H 50/00; G16H 50/20

(57) ABSTRACT

There are several data attributes available for a patient in electronic medical records, including vitals, medications, symptoms, notes from clinical encounters, and other biographical and demographic information. Various machine learning techniques can be leveraged to extract valuable insights from this data. However, it can be challenging to train machine learning algorithms on a large volume of labeled data in a supervised setting. As a solution to such deficiencies in supervised approaches, neural networks can be trained in an unsupervised environment, wherein autoencoders learn lower dimensional representations of the attributes. Once trained, the neural networks can effectively predict input features, detect anomalies, and forecast time series information.

### BACKGROUND

In general, machine learning involves the construction or generation of algorithms that can learn from data and perform complex calculations. These algorithms are often fine-tuned to perform specific tasks. In many instances, to effectively fine-tune algorithms for specific tasks, the algorithms will be trained using training data to aid the algorithms in learning more effectively. One approach to training machine learning algorithms is to implement a supervised learning technique whereby training data is enriched with labels and/or annotated. However, supervised learning is resource intensive, such that implementing a supervised approach may not be feasible in all circumstances.

For example, machine learning software solutions related to the evaluation and manipulation of medical records and patient information would require training data that is voluminous and includes numerous data types, and would further require many hours of manual labeling. Implementing a supervised learning technique that would require manual labeling, annotation, and consistent manual monitoring of the training process would be computationally and monetarily cost prohibitive. Indeed, conventional machine learning solutions in the medical industry are historically challenging to develop and scale, given the large volume of labeled data and task specific supervised learning that is required to bring these systems into existence. Accordingly, there is a need for systems and methods that overcome the deficiencies of prior art systems by enabling machine learning algorithms to learn and scale in an unsupervised environment.

### SUMMARY

There are several data attributes available for a patient in a typical electronic medical record, including vitals, medications, symptoms, notes from clinical encounters, and other biographical/demographic information. In order to use these attributes effectively, neural networks typically learn their lower dimensional representations called embeddings. These embeddings can be thought of as a mapping of categorical attributes to a vector of numbers that has an intrinsic semantic value. Data points that are closer to each other in the embedding are semantically similar. Embeddings learned for one task can be reused for another because the representation of attributes/features are useful regardless of the end task/objective.

As discussed, it can be challenging to train a machine learning algorithm and related features (e.g., a useful embedding) without a large volume of labeled data in a supervised setting. Therefore, as opposed to training a machine learning algorithm on medical records and patient data in a supervised setting, the instant solution overcomes the deficiencies in the prior art by training autoencoders in an unsupervised learning environment.

In particular, the system and methods described herein provide novel architecture and techniques for training autoencoders to predict patient vital information, detect anomalies in medical data, and conduct time series forecasting. These techniques discussed herein overcome the deficiencies of such conventional approaches by at least configuring encoders for the specific type of data feature being ingested, cross-leveraging embeddings across separate machine learning tasks and domains, concatenating disparate embedding vectors into one singular vector, and training autoencoders on data/features with known characteristics. These novel solutions may additionally include controlling the frequency in which medical records and patient data are received, such that real-time medical interventions may be generated.

In one embodiment, a system, a method, and a non-transitory computer-readable medium may respectively be configured, implement a process, and store instructions for: receiving one or more features from a user device as input for an autoencoder, wherein the one or more features includes vitals information; converting, by one or more deep neural network encoders, the one or more features into one or more latent embeddings; concatenating the one or more latent embeddings into a singular patient embedding; and reconstructing, by a decoder, the vitals information as output from the autoencoder using the singular patient embedding.

In another embodiment, a system, a method, and a non-transitory computer-readable medium may respectively be configured, implement a process, and store instructions for: receiving one or more features from a user device as input for an autoencoder; converting, by one or more deep neural network encoders, the one or more features into one or more latent embeddings; reconstructing, by a decoder, the one or more latent embeddings into reconstructed input, wherein the reconstructed input is representative of the one or more features; determining a reconstruction error by comparing the reconstructed input to the one or more features; and determining whether the one or more features is an anomaly by comparing the reconstruction error to a predetermined threshold.

In another embodiment, a system, a method, and a non-transitory computer-readable medium may respectively be configured, implement a process, and store instructions for: receiving one or more features from at least one user device as input for an autoencoder; converting, by one or more deep neural network encoders, the one or more features into one or more latent embeddings; reconstructing, by a decoder, the one or more latent embeddings into reconstructed input, wherein the reconstructed input is representative of the one or more features; and predict vital time series data associated with at least one user device based on the reconstructed input.

### BRIEF DESCRIPTION OF THE FIGURES

FIG. 1 illustrates a computing environment, according to various embodiments of the present disclosure.

FIG. 2 illustrates autoencoder architecture, according to various embodiments of the present disclosure.

FIG. 3 illustrates autoencoder architecture for generating predictions, according to various embodiments of the present disclosure.

FIG. 4 illustrates autoencoder architecture for detecting anomalies, according to various embodiments of the present disclosure.

FIG. 5 illustrates autoencoder architecture for time series forecasting, according to various embodiments of the present disclosure.

FIG. 6 illustrates a block diagram for a computing device, according to various embodiments of the present disclosure.

### DETAILED DESCRIPTION

Embodiments of the present disclosure relate to systems and methods for training and configuring autoencoders for generating predictions, detecting anomalies, and time series forecasting. The implementation of these novel concepts may include, in one respect, training one or more autoencoders with training data including known data features and configured encoders to ingest specific data types. Additionally, these novel concepts may include cross-leveraging embeddings across one or more machine learning domains and configuring machine learning/autoencoder architecture, such that an autoencoder(s) is specifically configured to implement one or more domain specific tasks.

The disclosed principles are described with reference to electronic health records and machine learning solutions within the medical industry, however, these principles may apply to any type of machine learning domain, wherein autoencoders can be trained and configured to perform a specific task. Accordingly, the disclosed principles are not limited to use with medical records and data.

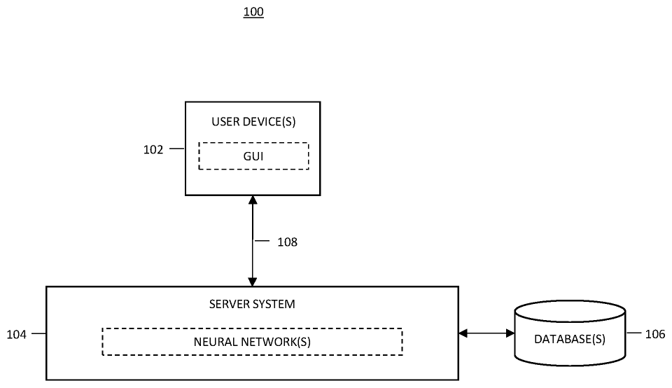

Referring to FIG. 1, computing environment 100 may be configured to implement a neural network for learning patient embeddings with autoencoders. Computing environment 100 may include one or more user device(s) 102, a server system 104, and one or more databases 106 communicatively coupled to the server system 104. The user device(s) 102, server system 104, and database(s) 106 may be configured to communicate through network 108.

In one or more embodiments, user device(s) 102 is operated by a user (e.g., a patient, a clinician, and/or engineer). User device(s) 102 may be representative of mobile phones, smart phones, tablet computers, laptop computers, desktop computers, patient devices, server computers or any other computing device configured to capture, receive, store and/or disseminate any suitable data. Users may include, but are not limited to, individuals such as, for example, individuals, patients, clinicians, companies, prospective clients, and or customers of an entity associated with server system 104, such as individuals who have received and or monitor patient health information and are utilizing the services of, or consultation from, an entity associated with server system 104.

In some embodiments, a user device(s) 102 includes non-transitory memory, one or more processors including machine readable instructions, a communications interface which may be used to communicate with the server system (and, in some examples, with the database(s) 106), a user input interface for inputting data and/or information to the user device and/or a user display interface for presenting data and/or information on the user device. In some embodiments, the user input interface and the user display interface are configured as an interactive graphical user interface (GUI). The user device(s) 102 are also configured to provide the server system 104, via the interactive GUI, input information (e.g., patient information (e.g., vitals data, medicine information, biographical information, and demographic information) and documents such as patient records, patient charts, clinician notes, and diagnoses) for further processing. In some embodiments, the interactive GUI is hosted by the server system 104 or provided via a client application operating on the user device. In addition, the interactive GUI may include multiple distinct regions where results can be provided, feedback can be inputted, and clinical documents can be created, updated, and revised. In some embodiments, a user operating the user device(s) 102 may query server system 104 for information related to a received patient health information/documents (e.g., patient vitals information/a clinical document).

Server system 104 hosts, stores, and operates machine learning neural network architecture for implementing one or more machine learning domain specific tasks. For example, server system 104 may include one or more autoencoders for generating predictions, detecting anomalies, and time series forecasting.

The neural network may automatically receive one or more features from a user device(s) 102 as input for an autoencoder, wherein the one or more features includes: patient vitals, patient medicine information, patient symptom information, clinician notes regarding a patient, and patient biographical and demographic information. In response to receiving the one or more features, at least one autoencoder (including one or more deep neural network encoders) within the neural network may convert the one or more features into one or more latent embeddings. Then at least one autoencoder may concatenate the embeddings for each feature of the one or more features into a singular patient embedding. One or more decoders within the at least one autoencoder may decode the singular patient embedding and reconstruct the one or more features as output from the autoencoder to predict, for example, patient vitals information.

In another embodiment, at the decoding phase, instead of concatenating patient embeddings, the decoder reconstructs the one or more features to generate a reconstruction error and subsequently determines if anomalies exist in the one or more features.

In another embodiment at the decoding phase, instead of concatenating patient embeddings or generating reconstruction error for anomaly detection, the decoder may reconstruct the one or more features to conduct time series forecasting.

In another embodiment, server system 104 may be configured to receive training data including known features, that, when ingested by an autoencoder, efficiently trains it to perform one or more machine learning domain specific tasks.

The server system 104 may further generate instructions for displaying information related to one or more machine learning domain specific tasks via a GUI that operates on the user device(s) 102. The server system 104 may be further configured to implement two-factor authentication, Secure Sockets Layer (SSL) protocols for encrypted communication sessions, biometric authentication, and token-based authentication. The server system 104 may include one or more processors, servers, databases, communication/traffic routers, non-transitory memory, modules, and interface components.

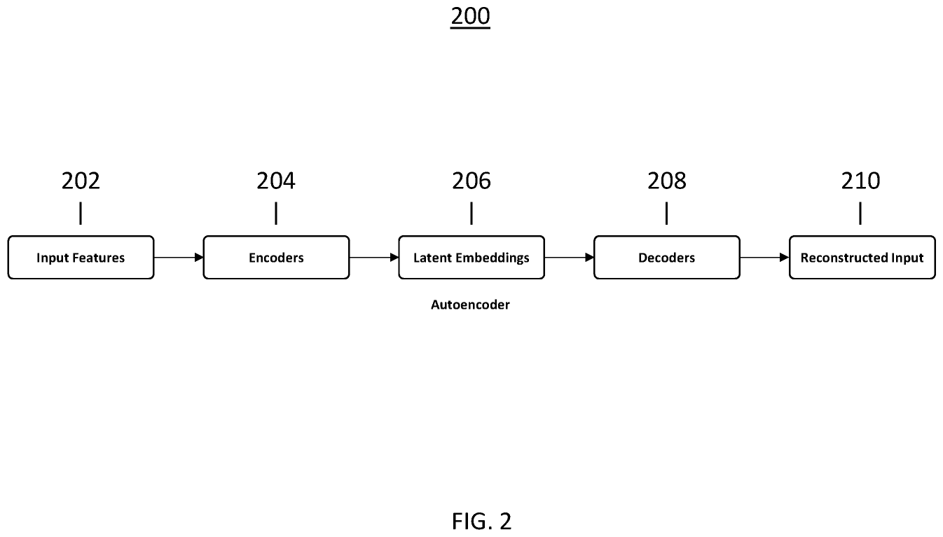

Database(s) 106 may be locally managed and/or a cloud-based collection of organized data stored across one or more storage devices and may be complex and developed using one or more design schema and modeling techniques. In one or more embodiments, the database system may be hosted at one or more data centers operated by a cloud computing service provider. The database(s) 106 may be geographically proximal to or remote from the server system 104 configured for data dictionary management, data storage management, multi-user access control, data integrity, backup and recovery management, database access language application programming interface (API) management, and the like. The database(s) 106 are in communication with the server system 104 and the user device(s) 102 via network 108. The database(s) 106 stores various data, including training data for one or more autoencoders. In some instances, database(s) 106 can be modified via queries initiated by users operating user device(s) 102. In one or more embodiments, various data in the database(s) 106 will be refined over time to improve/teach one or more autoencoders, as discussed below with respect to FIGS. 2-5. In one or more embodiments, database(s) 106 additionally stores historical training data and clinical/medical/patient information used by various components in computing environment 100. Additionally, the database system may be deployed and maintained automatically by one or more components shown in FIG. 1.

Network 108 is any suitable network, including individual connections via the Internet, such as cellular or Wi-Fi networks. In some embodiments, network 108 connects terminals, services, and mobile devices using direct connections, such as radio frequency identification (RFID), near-field communication (NFC), Bluetooth™, low-energy Bluetooth™ (BLE), Wi-Fi™, ambient backscatter communication (ABC) protocols, Universal Serial Bus (USB), wide area network (WAN), local area network (LAN), or the Internet. Because the information transmitted may be personal or confidential, security concerns may dictate that one or more of these types of connection be encrypted or otherwise secured. In some embodiments, however, the information transmitted may be less personal and, therefore, the network connections may be selected for convenience over security.

For example, network 108 may be the Internet, a private data network, virtual private network using a public network, and/or other suitable connection(s) that enables components in computing environment 100 to send and receive information between the components of computing environment 100.

FIG. 2 illustrates autoencoder architecture 200, according to various embodiments of the present disclosure. The autoencoder architecture 200 may include encoders 204 and decoders 208 to, in one respect, predict received input data by converting the input into lower dimensional latent space representation (i.e., embedding) and reconstruct the input data using the lower dimensional latent space representation. The autoencoders may be one or more of a sparse autoencoder, convolutional autoencoder, variational autoencoder, denoising autoencoder, contractive autoencoder, deep autoencoder, undercomplete autoencoder, and the like.

As depicted in FIG. 2, autoencoder architecture 200 may be configured to receive one or more input features 202 (e.g., patient vital information, patient medicine information, patient symptom information, patient/clinician notes, patient biographical/demographic information) as input. The encoders 204 may receive the input and encode it by converting it into a lower dimensional latent space representation (i.e., embedding) 206. The lower dimensional latent space representation 206 may be a vector. The decoders 208 may be configured to generate reconstructed input 210 based on the lower dimensional latent space representation 206 in an attempt to reconstruct the initial input features 202.

Autoencoder architecture 200 may be trained via unsupervised training techniques. In some instances, certain characteristics (e.g., labels or features) of the training data may be known, such that evaluation of the reconstructed input 210 in comparison to the input features 202 may be a computationally efficient process. For example, an autoencoder may be trained with training data with vital information (such as, historical patient blood pressure data) where the data values associated with vital information are known at the outset. When the autoencoder generates reconstructed input, given that the initial vital information was known, during downstream evaluation of the reconstructed input, it may be more readily apparent how accurate the autoencoder is at reconstructing the initial input and, accordingly, how the autoencoder may need to be modified in order become more accurate.

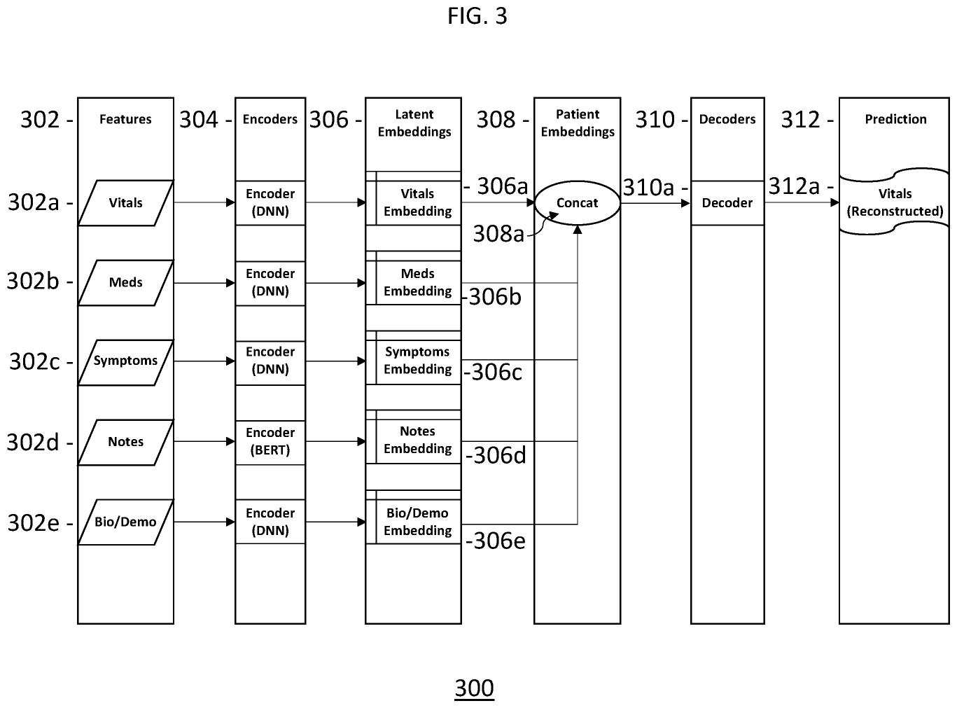

Notably, the reconstruction process autoencoder architecture 200 is configured to perform may be fundamental to one or more of the autoencoder architectures discussed in FIGS. 3-5.

FIG. 3 illustrates autoencoder architecture for generating predictions 300, according to various embodiments of the present disclosure. As discussed in relation to the autoencoder in FIG. 2, the autoencoder architecture for generating predictions 300 may include encoders 304 and decoders 310. The autoencoder architecture for generating predictions 300 may be configured to implement a method for receiving one or more features 302 as input and reconstructing the one or more features 302 for real-time production or training purposes. In a non-limiting capacity, the one or more features 302 may include patient vitals information 302a, patient medicine information 302b (e.g., prescriptions), patient symptoms information 302c, notes 302d (e.g., patient or clinician notes regarding a patient), and/or patient biographical/demographic information 302e. Notably, the one or more features 302 may be of various different data types. Some of the features of the one or more features 302 may be numerical, for example, patient vitals information 302a may be numerical data that can be normalized). Other features may be categorical data, for example, patient medicine information 302b and patient symptoms information 302c may be categorical. Notes 302d may be text and in some instances patient symptoms information 302c may be of text data type (instead of, or in addition to, being a categorical data type). Patient biographical/demographic information 302e may consist of both numerical and categorical data types.

The one or more features 302 may be respectively transmitted to corresponding encoders 304. The encoders 304 may be specifically configured to handle the data type associated with the one or more features 302. For example, the encoder that receives patient vitals information 302a may be specifically configured to encode numerical data related to patient vitals (e.g., heart rate, blood pressure, and the like). In a non-limiting capacity, encoders 304 may be deep neural network encoders and in response to receiving the one or more features 302, the encoders 304 may convert the one or more features 302 into lower dimensional latent space representations 306 (i.e., latent embeddings). For example, regardless of each features' original data type, each of the one or more features 302 may be converted into its own respective embedding (i.e., vector). For example, patient vitals information 302a may be converted into patient vitals embeddings 306a. Similar conversions may be done to generate patient medicine embeddings 306b, patient symptoms embeddings 306c, notes embeddings 306d, and patient biographical/demographic information 306e. Notably, the lower dimensional latent space representations 306, in some instances, can be the vector representation of the semantic meaning of the one or more features 302.

The lower dimensional latent space representations 306 may then be concatenated 308a to form a singular patient embedding 308. Here, the lower dimensional latent space representations 306 may be aggregated and combined to form a singular patient embedding 308 (e.g., a vector). The singular patient embedding 308 can be indicative of a holistic representation of a patient's state (e.g., health) at a given point in time.

The decoders 310 may receive the singular patient embedding 308, wherein it is configured to reconstruct one or all of the one or more features 302 based on the singular patient embedding 308. For example, the now concatenated singular patient embedding 308 may be passed to decoder 310a to reconstruct one or more of the one or more features. In one instance, despite the singular patient embedding including vectors related to each of the one or more features 302, the decoders 310 may be configured to only reconstruct the patient vitals information 302a, such that a prediction 312 including reconstructed patient vitals 312a is generated.

The autoencoder architecture for generating predictions 300, in conjunction with one or more components interacting with computing environment 100, may generate a reconstruction error, which is the difference between initial input vectors (i.e., vector representation of the one or more features 302/patient vitals information 302a) and output generated by decoders 310 (i.e., predictions 312/reconstructed patient vitals 312a). In the event autoencoder architecture for generating predictions 300 is being trained, one objective would be to reduce the reconstruction error in order to improve the accuracy of decoder 310 reconstructed input.

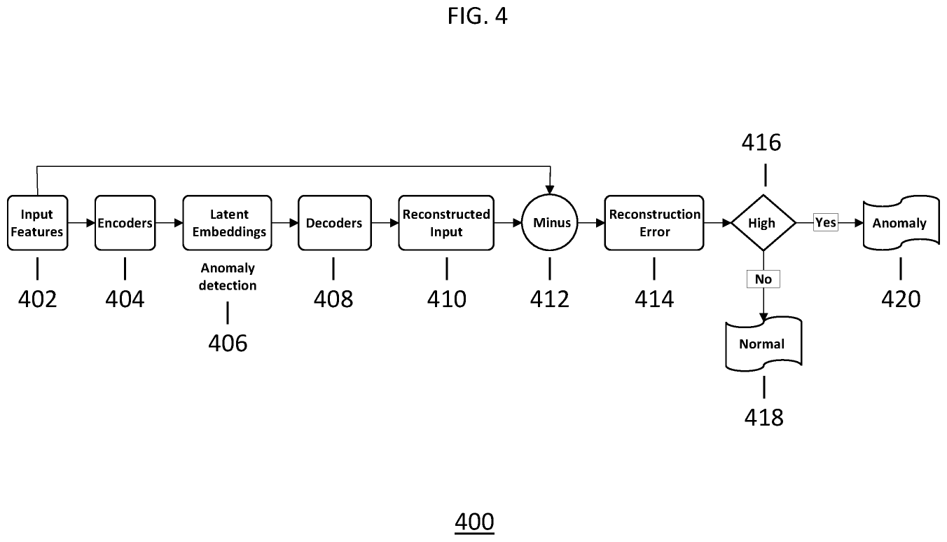

FIG. 4 illustrates autoencoder architecture for detecting anomalies 400, according to various embodiments of the present disclosure. As discussed in relation to the autoencoder in FIG. 2, autoencoder architecture for detecting anomalies 400 may include encoders 404 and decoders 408. The autoencoder architecture for detecting anomalies 400, may be configured to implement a method for receiving input features 402 and reconstructing the input features 402 for real-time production or training purposes. In a non-limiting capacity, the input features 402 may include patient vitals information, patient medicine information, patient symptoms information, notes (e.g., patient or clinician notes regarding a patient), and/or patient biographical/demographic information, as discussed in relation to FIG. 3.

The input features 402 may be respectively transmitted to corresponding encoders 404. The encoders 404 may be specifically configured to handle the data type associated with the input features 402. In one instance, input features 402 may be training data and in other instances input features 402 may be real-time production input for inference. In a non-limiting capacity, encoders 404 may be deep neural network encoders and in response to receiving the input features 402, the encoders 404 may convert the input features 402 into lower dimensional latent space representations 406 (i.e., latent embeddings). For example, regardless of each features' original data type, each of the input features 402 may be converted into its own respective embedding (i.e., vector). Notably, the lower dimensional latent space representations 406, in some instances, can be the vector representation of the semantic meaning of the input features 402.

The decoders 408 may receive the lower dimensional latent space representations 406, wherein the decoders 408 are configured to reconstruct one or all of the input features 402, by generating reconstructed input 410. The reconstructed input 410 may be compared to the input features 402 (i.e., the lower dimensional latent space representations 406/vectors). The comparison, in one instance may involve subtracting 412 the vector associated with the reconstructed input 410 from the vector associated with the input features 402, in order to generate reconstruction error 414. Here, the reconstruction error 414 may be unique to the particular type of input feature being analyzed. For example, patient vitals information may have a reconstruction error 414 that is different from patient medicine information reconstruction error 414.

The reconstruction error 414 may further be compared against a (predetermined) threshold value (e.g., a predetermined number or known variable). Architecture for detecting anomalies 400, in conjunction with one or more components interacting with computing environment 100, may determine/detect if anomaly 416 exists. For example, if the reconstruction error 414 meets or exceeds a predetermined threshold, it may be determined the reconstruction error 414 is high and therefore is an anomaly 420 relative to the data (i.e., training data) architecture for detecting anomalies 400 has been trained to process. In the alternative, if the reconstruction error 414 meets or is beneath a predetermined threshold, it may be determined the reconstruction error 414 is normal 418 relative to the data (i.e., training data) architecture for detecting anomalies 400 has been trained to process. Notably, the threshold value could be customizable and unique to the specific input feature being analyzed to ensure that the threshold value is specifically tailored to serve as trigger for accurately detecting anomalies for the specific input feature.

In a non-limiting example, autoencoder architecture for detecting anomalies 400 may be trained with four continuous weeks of one or more patients' vitals information (e.g., patient blood pressure data). The vitals information may be normalized and fed to autoencoder architecture for detecting anomalies 400 as input where an encoder would convert the vitals information into latent patient embeddings. A decoder would generate reconstructed input in an attempt to recreate the vitals information input. Subsequently, the reconstructed input (i.e., vector) would be compared (e.g., subtracted) from the vitals information input (i.e., patient embeddings) to generate a reconstruction error that is unique to the vitals information (e.g., patient blood pressure data) being processed. The reconstruction error would then be compared to a predetermined threshold value, which has an assigned value that has been determined to accurately serve as a trigger for detecting anomalies for vitals information. Here, if it is determined that the reconstruction error exceeds the predetermined threshold value, then an anomaly has been detected. For example, in a live-production environment, an anomaly could suggest that a patient's vitals (e.g., blood pressure) are outside of a normal range. In response, one or more downstream actions may be taken by a clinician to address the detected anomaly. In the alternative, it could be determined that the reconstruction error is beneath the predetermined threshold value and therefore the patient's vitals are within normal range.

In a training environment, detecting an anomaly could serve as an indication as to whether autoencoder architecture for detecting anomalies 400 is well-configured/trained to accurately detect anomalies for a specific feature. For example, given that the training data (e.g., four weeks of patient vitals information/blood pressure data) for autoencoder architecture for detecting anomalies 400 may be known, and instance where autoencoder architecture for detecting anomalies 400 is fed patient medicine information (assuming patient medicine information would generate different embeddings and reconstruction errors than vitals information) and generates a reconstruction error that exceeds a predetermined threshold value for vitals information, would signal that autoencoder architecture for detecting anomalies 400 may be accurately detecting anomalies. In the alternative, if a reconstruction error associated with patient medicine information did not trigger an anomaly being detected against a predetermined threshold value associated with vitals information, the failure to detect an anomaly may indicate that autoencoder architecture for detecting anomalies 400 may not accurately detect anomalies and therefore may require further training or modification.

Notably, vitals information may be received from user device(s) 102 according to certain frequencies set by users and or entities associated with server system 104. As such, the frequency by which vitals information (or other feature information) is received from user device(s) 102 may be modified in response to an anomaly being detected (or not being detected) or in response to determining that the architecture for detecting anomalies 400 would improve performance with modified reconstruction error/predetermined threshold values.

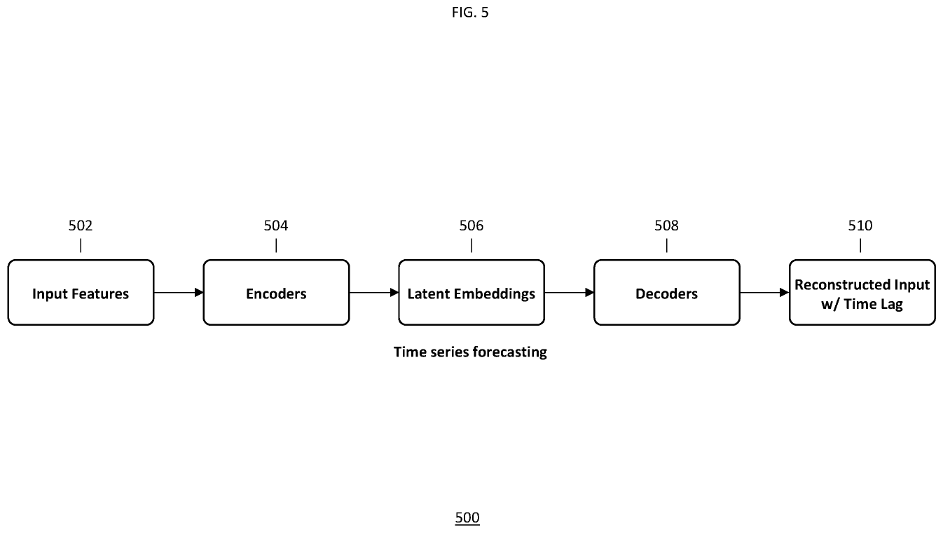

FIG. 5 illustrates autoencoder architecture for time series forecasting 500, according to various embodiments of the present disclosure. As discussed in relation to the autoencoder in FIG. 2, autoencoder architecture for time series forecasting 500 may include encoders 504 and decoders 508. The autoencoder architecture for time series forecasting 500 may be configured to implement a method for receiving input features 502 and reconstructing the input features 502 for time-series forecasting. In a non-limiting capacity, the input features 502 may include historical data (e.g., 4-5 previous weeks of patient vitals information, patient medicine information, patient symptoms information, notes (e.g., patient or clinician notes regarding a patient), and/or patient biographical/demographic information), as discussed in relation to FIG. 3.

The input features 502 may be respectively transmitted to corresponding encoders 504. The encoders 504 may be specifically configured to handle the data type associated with the input features 502. In one instance, input features 502 may be training data and in other instances input features 502 may be real-time production input. In a non-limiting capacity, encoders 504 may be deep neural network encoders and in response to receiving the input features 502, the encoders 504 may convert the input features 502 into lower dimensional latent space representations 506 (i.e., latent embeddings). In one instance, the encoders 504 step through the input features 502 including time steps related to patient vitals information. For example, the encoders 504 may encode an entire sequence of time series input time steps for each of the input features 502 and convert each sequence of time series input into its own respective embedding (i.e., a context vector). The context vector may represent an initial hidden state for the decoders 508 and may further include information to help the decoders 508 make accurate predictions.

The decoders 508 may receive the lower dimensional latent space representations 506, wherein the decoders 508 are configured to reconstruct one or all of the input features 502, by generating reconstructed input 510 with a time lag (e.g., time series predictions regarding the input features 502). In one instance, the decoders 508 are configured to step through the output time steps while reading from the context vector.

In a non-limiting example, autoencoder architecture for time series forecasting 500 might be trained on input features 502, for example, four weeks of previously captured patient blood pressure data. The encoders 504 may convert the patient blood pressure data into latent patient embeddings. The decoders 508 may receive the latent patient embeddings and future date features and lag features, wherein the lag features are previous weeks blood pressure data (e.g., the previous week relative to the date of the training, wherein the previous week is not included in the four weeks or previously captured patient blood pressure data). Based on this input, the decoders are configured to generate reconstructed input with time lag indicative of future states of the patient's blood pressure.

Notably, input features 502 may be received from user device(s) 102 according to certain frequencies set by users and or entities associated with server system 104. As such, the frequency by which input features 502 are received from user device(s) 102 may be modified in response to various time series forecasts or in response to determining that increasing/decreasing input features 502 frequency would improve the performance of autoencoder architecture for time series forecasting 500.

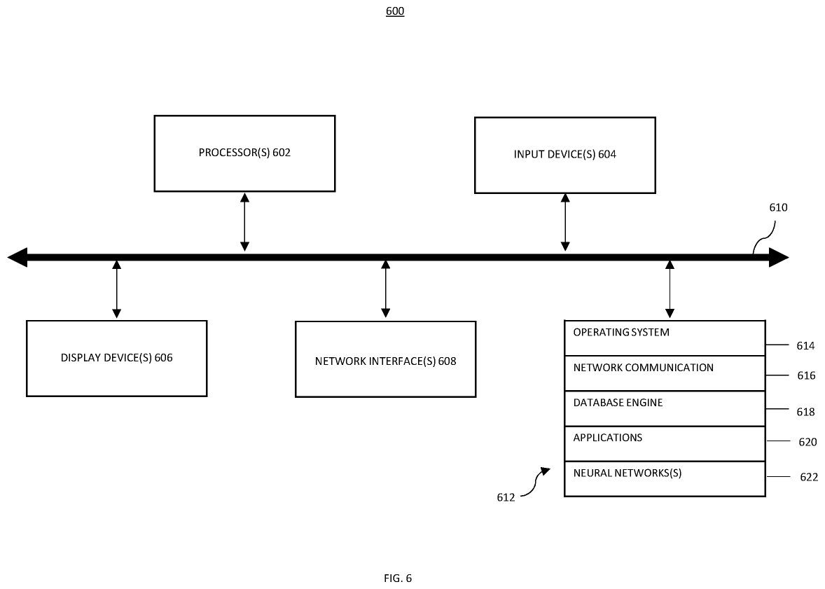

FIG. 6 illustrates a block diagram for a computing device, according to various embodiments of the present disclosure. For example, computing device 600 may function as server system 104. The computing device 600 may be implemented on any electronic device that runs software applications derived from compiled instructions, including without limitation personal computers, servers, smart phones, media players, electronic tablets, game consoles, and email devices. In some implementations, the computing device 600 may include processor(s) 602, input device(s) 604, display device(s) 606, network interfaces 608, and computer-readable medium(s) 612 storing software instructions. Each of these components may be coupled by bus 610, and in some embodiments, these components may be distributed among multiple physical locations and coupled by a network 108.

Display device(s) 606 may be any known display technology, including but not limited to display devices using Liquid Crystal Display (LCD) or Light Emitting Diode (LED) technology. Processor(s) 602 may use any known processor technology, including but not limited to graphics processors and multi-core processors. Input device(s) 604 may be any known input device technology, including but not limited to a keyboard (including a virtual keyboard), mouse, track ball, camera, and touch-sensitive pad or display. Bus 610 may be any known internal or external bus technology, including but not limited to industry standard architecture (ISA), extended industry standard architecture (EISA), peripheral component interconnect (PCI), peripheral component interconnect (PCI) Express, universal serial bus (USB), Serial advanced technology attachment (ATA), or FireWire. Computer-readable medium(s) 612 may be any non-transitory medium that participates in providing instructions to processor(s) 602 for execution, including without limitation, non-volatile storage media (e.g., optical disks, magnetic disks, flash drives, etc.), or volatile media (e.g., SDRAM, ROM, etc.).

Computer-readable medium(s) 612 may include various instructions for implementing an operating system 614 (e.g., Mac OS®, Windows®, Linux). The operating system may be multi-user, multiprocessing, multitasking, multithreading, real-time, and the like. The operating system may perform basic tasks, including but not limited to: recognizing input from input device(s) 604; sending output to display device(s) 606; keeping track of files and directories on computer-readable medium(s) 612; controlling peripheral devices (e.g., disk drives, printers) which can be controlled directly or through an input/output (I/O) controller; and managing traffic on bus 610. Network communications instructions 616 may establish and maintain network connections (e.g., software for implementing communication protocols, such as TCP/IP, HTTP, Ethernet, telephony).

Database processing engine 618 may include instructions that enable computing device 600 to implement one or more methods as described herein. Application(s) 620 may be an application that uses or implements the processes described herein and/or other processes. The processes may also be implemented in operating system 614. For example, application(s) 620 and/or operating system 614 may execute one or more operations to train autoencoders included in one or more neural networks 622 used in conjunction with one or more methods as described above.

The described features may be implemented in one or more computer programs that may be executable on a programmable system including at least one programmable processor coupled to receive data and instructions from, and to transmit data and instructions to a data storage system (e.g., database(s) 106), at least one input device, and at least one output device. A computer program is a set of instructions that can be used, directly or indirectly, in a computer to perform a certain activity or bring about a certain result. A computer program may be written in any form of programming language (e.g., Tensorflow, Keras, PyTorch, Cognitive Toolikit, Janusgraph, Gremlin, Sandbox, SQL, Objective-C, Java), including compiled or interpreted languages, and it may be deployed in any form, including as a stand-alone program or as a module, component, subroutine, or other unit suitable for use in a computing environment.

Suitable processors for the execution of a program of instructions may include, by way of example, both general and special purpose microprocessors, and the sole processor or one of multiple processors or cores, of any kind of computer. Generally, a processor may receive instructions and data from a read-only memory or a random-access memory or both. The essential elements of a computer may include a processor for executing instructions and one or more memories for storing instructions and data. Generally, a computer may also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

To provide for interaction with a user, the features may be implemented on a computer having a display device such as an LED or LCD monitor for displaying information to the user and a keyboard and a pointing device such as a mouse or a trackball by which the user can provide input to the computer.

The features may be implemented in a computer system that includes a back-end component, such as a data server, or that includes a middleware component, such as an application server or an Internet server, or that includes a front-end component, such as a client computer having a graphical user interface or an Internet browser, or any combination thereof. The components of the system may be connected by any form or medium of digital data communication such as a communication network. Examples of communication networks include, e.g., a telephone network, a LAN, a WAN, and the computers and networks forming the Internet.

The computer system may include clients and servers. A client and server may generally be remote from each other and may typically interact through a network. The relationship of client and server may arise by virtue of computer programs running on the respective computers and having a client-server relationship to each other.

One or more features or steps of the disclosed embodiments may be implemented using an API. An API may define one or more parameters that are passed between a calling application and other software code (e.g., an operating system, library routine, function) that provides a service, that provides data, or that performs an operation or a computation.

The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer will employ to access functions supporting the API.

In some implementations, an API call may report to an application the capabilities of a device running the application, such as input capability, output capability, processing capability, power capability, communications capability, etc.

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. For example, other steps may be provided, or steps may be eliminated, from the described flows, and other components may be added to, or removed from, the described systems. Accordingly, other implementations are within the scope of the following claims.

In addition, it should be understood that any figures which highlight the functionality and advantages are presented for example purposes only. The disclosed methodology and system are each sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims and drawings.

It is the applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

Although the present invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the scope of the appended claims. For example, it is to be understood that the present invention contemplates that, to the extent possible, one or more features of any embodiment can be combined with one or more features of any other embodiment.

1. A system comprising:
a server comprising one or more processors; and
a non-transitory memory, in communication with the server, storing instructions that when executed by the one or more processors, cause the one or more processors to implement a method comprising:
receiving one or more features from a user device as input for an autoencoder, wherein the one or more features includes vitals information;
converting, by one or more deep neural network encoders, the one or more features into one or more latent embeddings;
concatenating by exclusively appending the one or more latent embeddings together to generate a singular patient embedding; and
reconstructing, by a decoder, exclusively the vitals information as output from the autoencoder using the singular patient embedding, wherein the singular patient embedding comprises one or more latent embeddings associated with the one or more features.

2. The system of claim 1, further comprising, wherein the autoencoder is trained using one or more unsupervised training techniques.

3. The system of claim 1, further comprising wherein the autoencoder is trained on known training data with corresponding labels.

4. The system of claim 1, wherein concatenating the one or more latent embeddings into a singular patient embedding further comprises appending the one or more latent embeddings, such that the singular patient embedding consists of a least one singular vector.

5. The system of claim 1, wherein the singular patient embedding is indicative a holistic representation of a health state of one or more patients.

6. The system of claim 1, wherein reconstructing the vitals information includes predicting the vitals information included in the one or more features.

7. The system of claim 1, wherein the one or more features further includes one or more of: patient medicine information, patient symptom information, patient clinician notes, patient biographical, or demographic information.

8. A system comprising:
a server comprising one or more processors; and
a non-transitory memory, in communication with the server, storing instructions that when executed by the one or more processors, cause the one or more processors to implement a method comprising:
receiving one or more features from a user device as input for an autoencoder;
converting, by one or more deep neural network encoders, the one or more features into one or more latent embeddings;
reconstructing, by a decoder, the one or more latent embeddings into reconstructed input, wherein the reconstructed input is representative of the one or more features;
determining a reconstruction error by comparing the reconstructed input to the one or more features; and
determining whether the one or more features is an anomaly by comparing the reconstruction error to a predetermined threshold.

9. The system of claim 8, further comprising, wherein the autoencoder is trained using one or more unsupervised training techniques.

10. The system of claim 8, further comprising wherein the autoencoder is trained on known training data with corresponding labels.

11. The system of claim 8, wherein restructuring one or more latent embeddings further comprises predicting the one or more features.

12. The system of claim 8, wherein the one or more features further includes one or more of: patient medicine information, patient symptom information, patient clinician notes, patient biographical, or demographic information.

13. The system of claim 8, wherein each of the one or more latent embeddings has its own unique reconstruction error.

14-20. (canceled)

21. A system comprising:
a server comprising one or more processors; and
a non-transitory memory, in communication with the server, storing instructions that when executed by the one or more processors, cause the one or more processors to implement a method comprising:
receiving one or more features from a user device as input for an autoencoder, wherein the one or more features includes vitals information;
converting, by one or more deep neural network encoders, the one or more features into one or more latent embeddings;
generating a singular patient embedding consisting of the one or more latent embeddings concatenated by appending the one or more latent embeddings together; and
reconstructing, by a decoder, exclusively the vitals information as output from the autoencoder using the singular patient embedding, wherein the singular patient embedding comprises one or more latent embeddings associated with the one or more features.

22. The system of claim 21, further comprising, wherein the autoencoder is trained using one or more unsupervised training techniques.

23. The system of claim 21, further comprising wherein the autoencoder is trained on known training data with corresponding labels.

24. The system of claim 21, wherein concatenating the one or more latent embeddings into a singular patient embedding further comprises appending the one or more latent embeddings, such that the singular patient embedding consists of a least one singular vector.

25. The system of claim 21, wherein the singular patient embedding is indicative a holistic representation of a health state of one or more patients.

26. The system of claim 21, wherein reconstructing the vitals information includes predicting the vitals information included in the one or more features.

27. The system of claim 21, wherein the one or more features further includes one or more of: patient medicine information, patient symptom information, patient clinician notes, patient biographical, or demographic information.
